(19) United States
(12) Patent Application Publication
(10) Pub. No.: US 2013/0011434 A1
(43) Pub. Date: Jan. 10, 2013

(54) ADJUVANT DILUENTS FOR LIVE VACCINES FOR PIG DISEASES

(75) Inventors: Francois Bertrand, Paris (FR); Sebastien Deville, Paris (FR); Laurent Dupuis, Reims (FR)

(73) Assignee: SOCIETE D'EXPLOITATION DE PRODUITS POUR LES INDUSTRIES CHIMIQUES SEPPIC, Paris (FR)

(21) Appl. No.: 13/636,741

(22) PCT Filed: Mar. 15, 2011
(86) PCT No.: PCT/FR11/50514

§ 371 Date: Sep. 24, 2012

(30) Foreign Application Priority Data
Mar. 24, 2010 (FR) 1052114

(51) Int. Cl. A61K 39/12; A61P 31/14; A61P 37/04

(52) U.S. Cl. 424/204.1

(57) ABSTRACT

A method for preparing an injectable vaccine composition intended to combat porcine reproductive and respiratory syndrome (PRRS), includes at least the step in which: a) a live vaccine is mixed extemporaneously with an adjuvant diluent (AD). The adjuvant diluent is an oil-in-water-type emulsion or an oil-in-water-type microemulsion, or an aqueous solution including water and at least one inorganic salt selected from aluminum hydroxide, cerium nitrate, zinc sulfate, colloidal iron hydroxide or calcium chloride, salts of divalent or trivalent metals or sympathomimetic compounds.

The present invention relates to a method for preparing an injectable vaccine composition intended to combat porcine reproductive and respiratory syndrome (PRRS).

Porcine reproductive and respiratory syndrome (PRRS) is a viral infection which affects mainly domestic pigs, and which has also already been diagnosed in wild boars. The nature and seriousness of the problems depend on age, sex, and conditions in which the pigs are reared, and also on the strain of the virus.

The major damage caused by PRRS is essentially due to fertility problems and to pulmonary ailments specific to this disease.

A transient fever and respiratory problems due to pneumonia are the characteristic symptoms of PRRS. The lack of oxygen caused by the respiratory distress can cause cyanosis at the level of the ears, the stomach and the extremities. The pupils are swollen and an aqueous ocular discharge is observed.

On farms where the animals are fattened up, it is the low fattening yield due to pneumonia which causes most problems, whereas the fertility problems are naturally to the fore in breeding farms. In the latter, up to 50% of litters can undergo considerable losses.

Viruses, whether inhaled or swallowed, propagate first in the cells of the lung and the tonsils of the pharynx, and then throughout the body. The viruses can then be excreted via the most varied routes: nasal secretions, excrement, urine, semen, still-born fetuses. In pigpens, the disease propagates by direct contact with infectious material or by inhalation of infectious droplets. The propagation is much faster in the case of stress and high density of the animals. Since the infectious agent survives for a long time in an infected animal, pigs remain contagious even when cured. The virus can also be propagated by birds, insects, for instance flies, or by people (clothes, unwashed hands, equipment, etc.).

Vaccines, both live and inactivated, have been developed for use in sows and piglets.

Vaccination consists in inoculating the species to be protected with an amount of pathogen that has been killed (inactivated vaccine) or made nonpathogenic (live attenuated vaccine) in order to trigger a biological response in the host, protecting it during the subsequent occurrence of the disease.

Live vaccines are generally sufficiently effective so as not to require the use of adjuvants.

A vaccine adjuvant is an excipient which amplifies the biological response against the antigen with which it is combined. Mention will, for example, be made of aluminum hydroxide, and the oily adjuvants sold under the name Montanide™ by the company SEPPIC. These adjuvants are diverse in nature. They can just as easily consist of liposomes, of emulsions comprising at least one oily phase and at least one aqueous phase, of “Freund's” adjuvant type, or more commonly of water-insoluble inorganic salts. Among the inorganic salts used as vaccine composition adjuvants, mention may be made, for example, of aluminum hydroxide, cerium nitrate, zinc sulfate, colloidal iron hydroxide or calcium chloride. Aluminum hydroxide is the adjuvant most commonly used. These inorganic salts used as vaccine composition adjuvants are described, in particular, in the article by Rajesh K. Gupta et al., “Adjuvants, balance between toxicity and adjuvanticity”, Vaccine, vol. 11, Issue 3, 1993, pages 993-306.

In the case of PRRS, vaccines, both live vaccines and inactivated vaccines, have been developed for use in sows and piglets.

a) Inactivated vaccines are generally intended for vaccinating sows.

b) Live vaccines are especially used in piglets for pork meat, against respiratory problems, but recently a registration has also been obtained for the prevention of reproductive problems in sows.

Some live vaccines multiply for a long time in the lungs of the vaccinated piglets, and certain strains of live vaccines could cause a transplacental infection. Thus, the danger of vaccinating gestating sows which have never previously been in contact with the PRRS virus is understood. However, it is not rare for live vaccines to be used to create an infection with an attenuated virus, in order to induce collective immunity at a young age (piglets 3 to 4 weeks old) and/or to homogenize the immune status of non-gestating sows.

Among the live vaccines developed and sold for combating PRRS, mention may be made, for example, of the Amervac-PRRS/A3 vaccine sold by Hipra Lab., the Ingelvac PRRS Modified Live Virus vaccine sold by Boehringer Ingelheim, and the Porcilis PRRS vaccine sold by Intervet.

In the face of the occurrence of epidemics resistant to the use of conventional live vaccines, as in the case of PRRS, various adjuvant techniques have been tested in order to:
increase the strength of the protective response making it possible to provide a better level of protection;
prolong the duration of the protection conferred by a vaccine dose, providing longer-lasting protection of the animals on farms throughout their growth;
provide sufficient protection with a single treatment when two treatments were necessary in the absence of these immune response amplifiers. The saving made is therefore in terms of the number of doses to be injected (halved), the handling of the animals (labor) and the stress created during the handling of the animals (also halved);
obtain, with a lower antigenic dose, an efficacy equivalent to that conferred by a complete dose used without adjuvant. Thus, a vaccine production entity will, with the same productive capacity, be capable of producing a higher number of vaccine doses. Similarly, an existing packaging may be proposed for vaccinating a larger number of animals.

There is a need to develop diluents which also have the adjuvant function in order to improve the immune response as described above. These compositions are referred to as adjuvant diluents (ADs).

The principal difficulty encountered in the development of an AD is the ability of said adjuvant diluent (AD) to keep the live vaccine alive so that it retains its immunogenic properties.

An important element of the development of adjuvants for live vaccines lies in the specificity of the adjuvant formulations of not killing the live microorganisms constituting the vaccine antigens when they are brought into contact before injection. ADs exist on the market (such as, for example, tocopheryl acetate from the company Intervet included in Diluvac Forte®) and are recommended for certain live vaccines.

Furthermore, a live vaccine is known in French patent application FR 2 385 401.

However, this live vaccine is not prepared with adjuvant substances. It has up until now been taken as read that the greater immune response could be produced with a live vaccine, for example, by increasing the content of virus, or by using a more immunogenic strain.

According to the present invention, a method for preparing a live virus vaccine has been found, which is characterized in that the live vaccine is prepared by means of an adjuvant of the aqueous continuous phase emulsion type, for example a water-in-oil emulsion or microemulsion.

It has surprisingly been noted that the use of an oil-in-water (O/W) emulsion as “diluent” for live vaccines has a positive effect on the serological and immune response in vaccinated animals. In this oil-in-water emulsion, the external aqueous phase allows the vaccine (generally freeze-dried) to be easily dissolved.

Another aspect of the invention is that the use of the oil-in-water emulsion as adjuvant diluent for live vaccines causes a very high serological response in young animals but which still have maternal immunity. This surprising effect may be caused by the protective action of the emulsion, on the live virus, against neutralization by the antibodies present in the animal.

An objective of the present invention is to overcome all or some of the drawbacks of the prior art raised above.

To this end, a subject of the present invention is a method for preparing an injectable vaccine composition intended to combat porcine reproductive and respiratory syndrome (PRRS), comprising at least the step in which:

a) a live vaccine is mixed extemporaneously with an adjuvant diluent (AD); characterized in that said adjuvant diluent is an oil-in-water emulsion or an oil-in-water microemulsion, or an aqueous solution comprising water and at least one inorganic salt selected from aluminum hydroxide, cerium nitrate, zinc sulfate, colloidal iron hydroxide or calcium chloride, salts of divalent or trivalent metals or sympathomimetic compounds.

The term “diluent” is intended to mean a substance to be added to another in order to reduce the titer, the richness or the percentage thereof.

Moreover, embodiments of the invention may comprise one or more of the following characteristics:
method as defined above, characterized in that the adjuvant diluent (AD) comprises an adjuvant, an aqueous phase and, optionally, a polymer of the sodium polyacrylate family;
method as defined above, characterized in that the virus contained in said live vaccine is freeze-dried before step a);
method as defined above, characterized in that said adjuvant diluent is an oil-in-water emulsion comprising, for 100% of its weight:
   from 99% to 50% by weight of water, more particularly from 99% to 75% by weight of water, and even more particularly from 90% to 80% by weight of water;
   from 1% to 50% by weight of oily adjuvant, more particularly from 1% to 25% by weight of oily adjuvant, and even more particularly from 5% to 19% by weight of oily adjuvant;

method as defined above, characterized in that the adjuvant diluent also comprises from 0.25% to 5% by weight of polymers of the sodium polyacrylate family, more particularly from 0.2% to 4% by weight of polymers of the sodium polyacrylate family, and even more particularly from 0.3% to 3% by weight of polymers of the sodium polyacrylate family;
method as defined above, characterized in that said adjuvant diluent is an oil-in-water microemulsion comprising, for 100% of its weight:
   from 99% to 50% by weight of water, more particularly from 99% to 75% by weight of water, and even more particularly from 90% to 80% by weight of water;
   from 1% to 50% by weight of oily adjuvant, more particularly from 1% to 25% by weight of oily adjuvant, and even more particularly from 5% to 19% by weight of oily adjuvant;
method as defined above, characterized in that the adjuvant diluent also comprises from 0.25% to 5% by weight of polymers of the sodium polyacrylate family, more particularly from 0.2% to 4% by weight of polymers of the sodium polyacrylate family, and even more particularly from 0.3% to 3% by weight of polymers of the sodium polyacrylate family;
method as defined above, characterized in that said oily adjuvant comprises, for 100% of its weight: from 1% to 95% by weight of at least one mineral oil, more particularly from 10% to 90% by weight of at least one mineral oil, and even more particularly from 20% to 90% by weight of at least one mineral oil; and from 1% to 80% by weight of at least one surfactant, more particularly from 2% to 70% by weight of at least one surfactant, and even more particularly from 10% to 45% by weight of at least one surfactant;
method as defined above, characterized in that said adjuvant diluent is an aqueous solution comprising, for 100% of its weight:
   from 99.5% to 95.0% by weight of water, more particularly from 99.5% to 97.0% by weight of water, and even more particularly from 99.0% to 97.0% by weight of water;
   from 0.5% to 5.0% by weight of inorganic salts, more particularly from 0.5% to 3.0% by weight of inorganic salts, and even more particularly from 1.0% to 3.0% by weight of inorganic salts; and
method as defined above, characterized in that the aqueous solution comprises, for 100% of its weight:
   from 1% to 15% by weight, more particularly from 1% to 10% by weight, even more particularly from 1% to 5% by weight of at least one surfactant;
   from 1% to 25% by weight, more particularly from 1% to 10% by weight, even more particularly from 1% to 5% by weight of at least one mineral oil;
   from 40% to 97% by weight of water, more particularly from 50% to 95% by weight of water, and even more particularly from 75% to 90% by weight of water;
   from 0.1% to 5% by weight of inorganic salts, more particularly from 0.2% to 4% by weight of inorganic salts, and even more particularly from 0.5% to 3% by weight of inorganic salts.

The live vaccines are generally stored freeze-dried and must be re-suspended extemporaneously with an aqueous phase. The vaccine thus reconstituted must be used within hours following the addition of a diluent.

The mineral oils used to prepare the oily adjuvants are selected from the group consisting of hydrocarbon mineral oils obtained by distillation of oil and by implementing subsequent processing steps such as, for example, desulfurization, deasphalting, aromatic compound extraction, and wax extraction steps, and other finishing processing steps (mention may be made, for example, of oils of the Marcol 52, Marcol 82, Drakeol 5 and Drakeol 6 etc., type).

The surfactants present in the oily adjuvants are emulsifying surfactants which have a hydrophilic nature, characterized by an HLB value of between 8 and 19, more particularly between 8 and 15.

Such a surfactant may consist of an alkylpolyglycoside or a mixture of alkylpolyglycosides; saponins; lecithins; polyoxyethylated alkanols; polymers comprising polyoxyethylene and polyoxypropylene blocks; esters obtained by condensation of a fatty acid, advantageously a fatty acid liquid at 20° C., with a sugar, sorbitol, mannitol or glycerol. Said sugar may consist of glucose or sucrose or, preferably, mannitol. By way of preferred esters, mention may be made of esters of fatty acids, for instance oleic acid, stearic acid, palmitic acid or lauric acid, and of sorbitol or mannitol, obtained by esterification of the fatty acid with sorbitol or mannitol, or else by esterification with the products resulting from the anhydrization of the polyhydroxylated chain of sorbitol or of mannitol which cyclizes at position 1-4 or at position 2-6, or else by esterification with sorbitol or mannitol and with the products resulting from the anhydrization of the polyhydroxylated chain of sorbitol or of mannitol which cyclizes at position 1-4 or at position 2-6. As particularly preferred mannitol esters, mention may be made of mannitol oleates, mannitan oleates, ethoxylated mannitol oleates comprising 5 mol or 10 mol or 15 mol or 20 mol of ethylene oxide, and ethoxylated mannitan oleates comprising 5 mol or 10 mol or 15 mol or 20 mol of ethylene oxide. Polyethylene glycol, sorbitol or glycerol sugar ester derivatives may also be used. The other types of preferred surfactants consist of ethoxylated plant oils, for instance ethoxylated corn oils having between 3 mol and 40 mol of ethylene oxide, ethoxylated rapeseed oils having between 3 mol and 40 mol of ethylene oxide, and ethoxylated castor oils having between 3 mol and 60 mol of ethylene oxide.

### EXAMPLES

The compatibility of the adjuvant formulae with the viability of freeze-dried vaccines is related to the composition of this adjuvant formula and to the amount at which it is used. Biocompatible constituents combined in proportions providing good implementation and an adjuvant capacity were selected and this selection was then evaluated in quantitative study protocols. The adjuvant effect was then evaluated on pig livestock brought into contact with the disease; various groups having received various vaccines; the protective effect was measured by quantification:
of the specific anti-PRRS antibody titers in blood samples taken after vaccination;
of pulmonary lesions characteristic of the bacterial superinfections associated with the pathogenesis of PRRS;

of the persistence of the fever following the virulent challenge.

Study 1: Viability of the Live Vaccine

The quantification of the virucidal effect is carried out according to a method which makes it possible to quantify the amount of virus remaining alive for a period of time following redilution suitable for the use and for the regulatory constraints (such as, for example, the European or US pharmacopeia). The live vaccine, freeze-dried material is brought into contact with the AD containing various amounts of various adjuvants, for a fixed period of time. The quantification of virus after the bringing into contact makes it possible to determine the viricidal nature of the AD.

The results of the virucidal activity test are compared with the viability, under the same conditions, of a suspension of the freeze-dried material with pure water.

An AD is judged to be nonvirucidal if the ratio of the final concentration to the initial concentration of virus is less than or equal to 7. In reality less than 0.7 in our case, since the values are expressed as logarithm.

a) Products which are Subject to the Study

The adjuvants (ADJ) used to prepare the oily ADs have the following compositions by weight, for 100% of their weight:

ADJ 1:

Mannitan oleate=40% by weight

Marcol 52=60% by weight

ADJ 2:

Sorbitan oleate=30% by weight

Marcol 52=70% by weight

ADJ 3:

Mannitan oleate=15% by weight

Marcol 52=85% by weight

ADJ 4:

Sorbitan oleate=15% by weight

Marcol 52=85% by weight

Various Adjuvant Diluents Tested (Proportions by Weight of Each of the Constituents for 100% of the Weight of AD):

TABLE 1

| | Adjuvant diluent | | | | | | | |
|---|---|---|---|---|---|---|---|---|
| | AD11 | AD12 | AD13 | AD14 | AD15 | AD16 | AD17 | AD18 |
| Water | 95% | 90% | 85% | 80% | 75% | 70% | 50% | 45% |
| Adjuvant 1 | 5% | 5% | 15% | 15% | 25% | 25% | 50% | 50% |
| Polymer | 0% | 5% | 0% | 5% | 0% | 5% | 0% | 5% |

TABLE 2

| | Adjuvant diluent | | | | | | | |
|---|---|---|---|---|---|---|---|---|
| | AD21 | AD22 | AD23 | AD24 | AD25 | AD26 | AD27 | AD28 |
| Water | 95% | 90% | 85% | 80% | 75% | 70% | 50% | 45% |
| Adjuvant 2 | 5% | 5% | 15% | 15% | 25% | 25% | 50% | 50% |
| Polymer | 0% | 5% | 0% | 5% | 0% | 5% | 0% | 5% |

TABLE 3

| | Adjuvant diluent | | | | | | | |
|---|---|---|---|---|---|---|---|---|
| | AD31 | AD32 | AD33 | AD34 | AD35 | AD36 | AD37 | AD38 |
| Water | 95% | 90% | 85% | 80% | 75% | 70% | 50% | 45% |
| Adjuvant 3 | 5% | 5% | 15% | 15% | 25% | 25% | 50% | 50% |
| Polymer | 0% | 5% | 0% | 5% | 0% | 5% | 0% | 5% |

TABLE 4

| | Adjuvant diluent | | | | | | | |
|---|---|---|---|---|---|---|---|---|
| | AD41 | AD42 | AD43 | AD44 | AD45 | AD46 | AD47 | AD48 |
| Water | 95% | 90% | 85% | 80% | 75% | 70% | 50% | 45% |
| Adjuvant 4 | 5% | 5% | 15% | 15% | 25% | 25% | 50% | 50% |
| Polymer | 0% | 5% | 0% | 5% | 0% | 5% | 0% | 5% |

TABLE 5

| | Adjuvant | | |
|---|---|---|---|
| | diluent | | |
| | AD51 | AD52 | AD53 |
| Water | 95% | 85% | 75% |
| Adjuvant | 0% | 0% | 0% |
| Polymer | 5% | 15% | 25% |

The polymer used in the preparation of the ADs above is a sodium polyacrylate, which can be in the form of a powder or else of an inverse latex such as that present in the composition sold under the name Montanide™ Gel.

The results of virucidal activity of the various adjuvant diluents are given in table 6.

TABLE 6
Virucidal effects of the adjuvant diluents tested

| | Virucidal effect according to US |
|---|---|
| Adjuvant diluent | pharmacopeia 9cfr113.35 |
| AD11 | 0.12 |
| AD12 | 0.08 |
| AD13 | 0.332 |
| AD14 | 0.34 |
| AD15 | 0.45 |
| AD16 | 0.52 |
| AD21 | 0.14 |
| AD22 | 0.11 |
| AD23 | 0.27 |
| AD24 | 0.35 |
| AD25 | 0.48 |
| AD26 | 0.58 |
| AD37 | 1.77 |
| AD38 | 1.82 |
| AD47 | 2.39 |
| AD48 | 2.19 |
| AD51 | 0.12 |
| AD52 | 0.08 |
| AD53 | 1.52 |

The results of the virucidal activity test of table 6 are compared with the viability, under the same conditions, of a composition resulting from the resuspension of the freeze-dried antigen material (the freeze-dried material in question is the freeze-dried PRRS virus). The amount of virus injected in the vaccination without adjuvant is 10⁴ TCID 50/dose of 2 ml (“tissue infective dose” 50%/dose of virus modifying 50% of the cells in standardized in vitro tests) with pure water.

Interpretation:

AD11, AD12, AD13, AD14, AD15, AD16, AD21, AD22, AD23, AD24, AD25 and AD26, comprising from 5% to 25% of adjuvant 1 and 2, and AD51 and AD52 comprising from 5% to 15% of polymers, are found to be nonvirucidal.

Study 2: Efficacy of the Adjuvanted Vaccines in Pigs

The pigs not contaminated with PRRS are vaccinated with various ADs while retaining the same freeze-dried live vaccine material (PRRS virus). This freeze-dried material corresponds to a commercial vaccine sold to be diluted in water.

Certain vaccines are prepared with only 50% of the viral dose, but in the presence of AD.

Groups of 10 pigs receive an injection of vaccine at the age of 4 weeks.

A virulent challenge by bringing into contact with a pathogenic PRRS virus is carried out at the age of three months. The animals are slaughtered three weeks after the virulent challenge. The efficacy of the vaccines is quantified by:
monitoring of the specific anti-PRRS antibody titers in the blood samples taken after vaccination;
the duration of the fever induced by the virulent challenge;
the lesions observed on the lungs after autopsy.

The adjuvant diluents AD I and AD II were prepared in order to be tested.

AD I (composition by weight):
Adjuvant 1 (ADJ 1)=15%
Water=85%
   AD II (composition by weight):
Adjuvant 1 (ADJ 1)=1%
Water=95%
Carbopol 971 (sodium polyacrylate)=4%

The results for the groups vaccinated with AD I (group I) and AD II (group II) were compared with the results obtained under the same conditions with a group vaccinated with a vaccine but which is in suspension in water without adjuvant (group T), a group which is not vaccinated but which is challenged (group NV) and a sentinel group, which receives neither vaccine nor virulence challenge, in order to verify that an epidemic does not disrupt the trial (group SE).

TABLE 7
Antibody titers 90 days after vaccination (before virulent challenge) and
animals having undergone seroconversion, as a function of vaccine groups

| | Vaccine groups | | | | | | |
|---|---|---|---|---|---|---|---|
| | | | | | | Control for | |
| | | | | | | virulent PRRS | |
| | Group II | Group I | Group T | Group II | Group I | virus (group NV) | Group SE |
| % of viral load | 100% | 100% | 100% | 50% | 50% | | |
| | antigen | antigen | antigen | antigen | antigen | | |
| Animals having | 10/10 | 10/10 | 10/10 | 10/10 | 10/10 | 0/10 | 0/5 |
| undergone | | | | | | | |
| seroconversion | | | | | | | |
| (i.e. having | | | | | | | |
| developed an | | | | | | | |
| immune | | | | | | | |
| response) | | | | | | | |
| Antibodies | 1.566 ± 0.255 | 1.735 ± 0.386 | 1.520 ± 0.404 | 1.768 ± 0.641 | 1.347 ± 0.976 | 0.010 ± 0.00 | 0.010 ± 0.00 |
| before vaccine | | | | | | | |
| (OD, ELISA) | | | | | | | |

Results of monitoring the specific anti-PRRS antibody titers in blood samples taken after vaccination:
   the results of antibody titers at 90 days after vaccination are given in table 7. All the vaccinated animals underwent seroconversion and the antibody titers are similar for all the groups, without any significant difference. The nonvaccinated sentinel and control groups are unaffected.
Results regarding the duration of the fever induced by the virulent challenge:
   at the time of the virulent challenge, a temperature peak is generally observed. Protected vaccinated animals generally return to a normal temperature more rapidly due to the immunity previously developed due to the vaccination.

TABLE 8
Average duration of fever (in days) after virulent challenge of
the groups of ten animals having received the various vaccines

| | Vaccine groups | | | | | | |
|---|---|---|---|---|---|---|---|
| | | | | | | Control for | |
| | | | | | | virulent PRRS | Group |
| | Group II | Group I | Group T | Group II | Group I | virus (group NV) | SE |
| % of viral | 100% | 100% | 100% | 50% | 50% | | |
| load | antigen | antigen | antigen | antigen | antigen | | |
| Average | 5.2 ± 2.3 | 4.1 ± 1.4 | 6.3 ± 2.8 | 6.2 ± 2.8 | 6.4 ± 2.5 | 10.7 ± 1.8 | 0 |
| duration of | | | | | | | |
| post-vaccine | | | | | | | |
| fever (days) | | | | | | | |

The nonvaccinated group (group NV) exhibits a fever duration of approximately 10 days.

Groups (I) and (II) vaccinated with 50% antigen and also the group vaccinated with the commercial control vaccine (group T) exhibit a fever duration of approximately 6 days.

Groups (I) and (II) vaccinated with 100% antigen exhibit a reduced fever duration of 4 days (group I) and of 5 days (group II).

The sentinel group exhibits no hyperthermia.
Results regarding the lesions observed on the lungs after autopsy:
   the amounts of lesions observed on the various lungs after autopsy are given in table 9.

TABLE 9
Pulmonary lesions induced by the virulent challenge on the various groups at slaughter

| | Vaccine groups | | | | | | |
|---|---|---|---|---|---|---|---|
| | | | | | | Control for | |
| | | | | | | virulent PRRS | |
| | Group II | Group I | Group T | Group II | Group I | virus (group NV) | Group SE |
| % of viral | 100% | 100% | 100% | 50% | 50% | | |
| load | antigen | antigen | antigen | antigen | antigen | | |
| Pulmonary | 2.63 ± 1.94 | 1.60 ± 1.72 | 12.04 ± 17.84 | 4.84 ± 8.18 | 5.58 ± 7.25 | 18.41 ± 22.79 | 0.44 ± 0.49 |
| lesions (%) | | | | | | | |

The nonvaccinated group (group NV) exhibits the highest amount of lesions, 18, with a standard deviation of 22, indicating substantial but heterogeneous lesions.

Group T, vaccinated with the commercial vaccine not comprising adjuvant, gives an amount of 12 with a standard deviation of 18.

Groups (I) and (II) vaccinated with a viral load at 50% antigen give amounts of approximately 5 with a standard deviation of 8.

Groups (I) and (II) vaccinated with a viral load at 100% antigen give the lowest scores (approximately 2 with a standard deviation of 2).

A low standard deviation indicates a good homogeneity of response.

In the case of groups (I) and (II), i.e. the groups vaccinated with, respectively, the adjuvant diluents AD I and AD II, the individual susceptibilities of the animals were reduced while harmonizing the levels of response. This point is very important for avoiding a situation where some infected animals maintain continuous foci of propagation by excretion of virus throughout rearing.

### CONCLUSION OF EXPERIMENTAL TESTS

These experimental results clearly demonstrate that the use of adjuvants of type 1 and 2, for preparing adjuvant diluents of type AD I and AD II (with or without polymer), improves the efficacy of the vaccine with respect to the criteria of duration of hyperthermia and protection of lung integrity; the results are very much better than a commercial vaccine using water as diluent instead of an AD containing adjuvant 1; the results obtained with only 50% of the antigen load, but with the ADs, are better than the commercial vaccine.

1. A method for preparing an injectable vaccine composition intended to combat porcine reproductive and respiratory syndrome (PRRS), comprising at least the step in which:
a) a live vaccine is mixed extemporaneously with an adjuvant diluent (AD); characterized in that said adjuvant diluent is an oil-in-water emulsion or an oil-in-water microemulsion, or an aqueous solution comprising water and at least one inorganic salt selected from aluminum hydroxide, cerium nitrate, zinc sulfate, colloidal iron hydroxide or calcium chloride, salts of divalent or trivalent metals or sympathomimetic compounds.

2. The method as claimed in claim 1, characterized in that the adjuvant diluent (AD) comprises an adjuvant, an aqueous phase and, optionally, a polymer of the sodium polyacrylate family.

3. The method as claimed in claim 1, characterized in that the virus contained in said live vaccine is freeze-dried before step a).

4. The method as claimed in claim 1, characterized in that said adjuvant diluent is an oil-in-water emulsion comprising, for 100% of its weight:
from 99% to 50% by weight of water, more particularly from 99% to 75% by weight of water, and even more particularly from 90% to 80% by weight of water;
from 1% to 50% by weight of oily adjuvant, more particularly from 1% to 25% by weight of oily adjuvant, and even more particularly from 5% to 19% by weight of oily adjuvant.

5. The method as claimed in claim 4, characterized in that the adjuvant diluent also comprises from 0.25% to 5% by weight of polymers of the sodium polyacrylate family, more particularly from 0.2% to 4% by weight of polymers of the sodium polyacrylate family, and even more particularly from 0.3% to 3% by weight of polymers of the sodium polyacrylate family.

6. The method as claimed in claim 1, characterized in that said adjuvant diluent is an oil-in-water microemulsion comprising, for 100% of its weight:
from 99% to 50% by weight of water, more particularly from 99% to 75% by weight of water, and even more particularly from 90% to 80% by weight of water;
from 1% to 50% by weight of oily adjuvant, more particularly from 1% to 25% by weight of oily adjuvant, and even more particularly from 5% to 19% by weight of oily adjuvant.

7. The method as claimed in claim 6, characterized in that the adjuvant diluent also comprises from 0.25% to 5% by weight of polymers of the sodium polyacrylate family, more particularly from 0.2% to 4% by weight of polymers of the sodium polyacrylate family, and even more particularly from 0.3% to 3% by weight of polymers of the sodium polyacrylate family.

8. The method as claimed in claim 4, characterized in that said oily adjuvant comprises, for 100% of its weight: from 1% to 95% by weight of at least one mineral oil, more particularly from 10% to 90% by weight of at least one mineral oil, and even more particularly from 20% to 90% by weight of at least one mineral oil; and from 1% to 80% by weight of at least one surfactant, more particularly from 2% to 70% by weight of at least one surfactant, and even more particularly from 10% to 45% by weight of at least one surfactant.

9. The method as claimed in claim 1, characterized in that said adjuvant diluent is an aqueous solution comprising, for 100% of its weight:
from 99.5% to 95.0% by weight of water, more particularly from 99.5% to 97.0% by weight of water, and even more particularly from 99.0% to 97.0% by weight of water;
from 0.5% to 5.0% by weight of inorganic salts, more particularly from 0.5% to 3.0% by weight of inorganic salts, and even more particularly from 1.0% to 3.0% by weight of inorganic salts.

10. The method as claimed in claim 9, characterized in that the aqueous solution comprises, for 100% of its weight:
from 1% to 15% by weight, more particularly from 1% to 10% by weight, even more particularly from 1% to 5% by weight of at least one surfactant;
from 1% to 25% by weight, more particularly from 1% to 10% by weight, even more particularly from 1% to 5% by weight of at least one mineral oil;
from 40% to 97% by weight of water, more particularly from 50% to 95% by weight of water, and even more particularly from 75% to 90% by weight of water;
from 0.1% to 5% by weight of inorganic salts, more particularly from 0.2% to 4% by weight of inorganic salts, and even more particularly from 0.5% to 3% by weight of inorganic salts.

11. The method as claimed in claim 2, characterized in that the virus contained in said live vaccine is freeze-dried before step a).
